(19) United States
(12) Patent Application Publication
(10) Pub. No.: US 2006/0286554 A1
(43) Pub. Date: Dec. 21, 2006

(54) Fluid container for electrohydrodynamic spray device and method of using same

(76) Inventor: Brian Graham, Dublin, OH (US)

Correspondence Address: DENNIS KILFOIL, 1362 MOONBEAM COURT NW, SALEM, OR, 97304, US

(21) Appl. No.: 10/541,684

(22) PCT Filed: Jan. 10, 2004
(86) PCT No.: PCT/US04/00555

§ 371 Date: Jan. 3, 2006

Related U.S. Application Data
(60) Provisional application No. 60/439,331, filed on Jan. 10, 2003.
(60) Provisional application No. 60/439,616, filed on Jan. 11, 2003.
(60) Provisional application No. 60/478,567, filed on Jun. 12, 2003.

(51) Int. Cl. C12Q 1/68

(52) U.S. Cl. 435/6

(57) ABSTRACT

A fluid container (6) for use with an electrohydrodynamic spray device (1) and method of using the device (1) are provided. The container (6) comprises at least one reservoir configured for holding a sprayable liquid, at least one interface (23) in fluid communication with the reservoir, at least one manifold (22) comprising at least one fluid path in fluid communication with the interface (23), and at least one nozzle (8). The nozzle (8) comprises at least one spray site in fluid communication with the fluid path. The device (1) is configured to engage the container (6). The container (6) can be configured such that when the container (1) is engaged with the device (1), the device (1) recognizes the sprayable liquid within the container (6) and automatically (or manually) adjusts certain pre-defined settings in accordance with the characteristics of the sprayable liquid.

The present invention relates to the production of an aerosol by means of electrohydrodynamics, and a device for the application of the electrically charged particles of material so produced. More particularly, the invention relates to a fluid container for an electrohydrodynamic spray device and method of using same for the application of chemical agents such as, for example, herbicides, fungicides, insecticides, acaricides, miticides, molluscicides, nematicides, rodenticides, plant desiccants, plant-growth regulators, etc. (pesticides in general), to a target.

The application of liquid formulations to crops, turfs, trees, and other plants as well as spraying of pesticides in and around the home using sprayers has been practiced for many years. More specifically, electrostatic sprayers have been developed to atomize pesticides for application on a target. For example, Coffee, U.S. Pat. No. 4,356,528, teaches an electrostatic sprayer to be used for ultra-low volume (“ULV”) spraying of pesticides. The sprayer includes multiple metal capillary tubes as spray nozzles, an insulating cover, and a field-intensifying ring electrode. Other examples of electrostatic sprayers include U.S. Pat. Nos. 4,376,514; 4,467,961; 4,470,550; 4,735,364; 4,476,515; 4,580,721; 4,629,164; and 6,105,571. However, no electrostatic sprayer system is known which allows for variations in spraying parameters in response to formulations/active ingredients sprayed through one or more nozzles. Prior to the present invention, electrostatic devices and fluid containers were instead tailored to the specific formulations or active ingredient being delivered. Accordingly, there is a recognized need for improvements in electrohydrodynamic sprayer design.

The present invention meets the above-mentioned need by providing a fluid container or cartridge for use with an electrohydrodynamic spray device, which device can produce a charged aerosol. As used herein, the terms fluid “container” and “cartridge” are intended to be interchangeable, and each can be defined as a vessel for holding a liquid formulation that is sprayed by the electrohydrodynamic device. Although the present invention is not limited to specific advantages or functionality, it is noted that the fluid container contributes to user safety by defining a secured container that prevents any interaction between the user of the electrohydrodynamic sprayer and the liquid formulation that is being sprayed. The design of the fluid container provides for quick and easy operator (“do-it-yourselfer”) change over of formulations and active ingredients. Also, the fluid container is configured for ease of clean up in all steps of operation and is relatively small in size, which minimizes waste. Moreover, the fluid container can be configured to be refillable, recyclable, or disposable following its use.

It is contemplated that the present invention has applications both inside and outside of homes as well as businesses or industrial environments. In addition, the scale of use of the present invention can reach from individual pest treatments (e.g., weeds), to targeted or specific areas of treatment, through commercial large-scale use. Various formulations and active ingredient fluids have different electrohydrodynamic spraying requirements (e.g., flow rate, voltage, etc.), which can be accommodated by the present invention either manually or automatically via a container/contents recognition feedback loop and control panel. Accordingly, the fluid container can be configured so that the electrohydrodynamic spray device automatically adjusts certain settings in response to particular formulations within the fluid container. This feature contributes to the ease of use of the present invention and assures the delivery of a quality spray, as well as a prescribed dose of a liquid formulation to an intended target. Moreover, the fluid container can incorporate security features such that the electrohydrodynamic spray device prevents the spraying of an unauthorized fluid formulation.

The fluid container of the present invention can also be configured so that the amount and expiration date of the liquid formulation within the container is recognized by the electrohydrodynamic device, which alerts the user when a new fluid container is needed. Accordingly, long-term storage of liquid formulations within the fluid container of the present invention are possible with no degradation of system operation and efficiency parameters. Moreover, the fluid container can be configured to automatically rinse the electrohydrodynamic nozzle and/or the container itself of a liquid formulation via a cleansing and/or pumping mechanism, which can be part of the container, the delivery device, or a separate element.

The fluid container can be provided in combination with varying nozzle/fluid/container configurations which provides means to use one electrohydrodynamic spray system or device in a variety of embodiments (e.g., wand, hand-held, cart-driven, etc.), with a variety of formulations or active ingredients, used in series with a common nozzle or set of multiple nozzles. As described with more particularity herein, the device is configured for easy insertion and removal of containers housing different formulations or active ingredients (in a variety of forms), each container having one or more formulation or active ingredient. Alternatively, the device can have ports (i.e., fluid paths, manifolds) for numerous containers to be inserted in a cartridge housing simultaneously. Moreover, means to ensure that the electrohydrodynamic spray device is not plugged or fouled by cross-contamination or aged material can be provided in a variety of ways (e.g., flush, purge, fluid, air, separate fluid paths, etc.), if necessary.

In accordance with one embodiment of the present invention, a fluid container for use with an electrohydrodynamic spray device is provided. The container comprises at least reservoir configured for holding a sprayable liquid, at least one interface in fluid communication with the reservoir, at least one manifold comprising at least one fluid path in fluid communication with the interface, and at least one nozzle comprising at least one spray site in fluid communication with the fluid path. The device is configured to engage the container. As such, the device and the container are each configured so that at least a portion of the container extends out from the device.

In accordance with another embodiment of the present invention, a fluid container for use with an electrohydrodynamic spray device is provided. The container comprises two or more separate reservoirs, each configured for holding a disparate sprayable liquid. The container also comprises two or more interfaces, each in fluid communication with one reservoir, at least one manifold comprising two or more separate fluid paths, each fluid path in fluid communication with one interface, and at least one nozzle. The nozzle comprises at least one spray site in fluid communication with one or more of the fluid paths.

In accordance with yet another embodiment, a fluid containment system for use with an electrohydrodynamic spray device is provided wherein the system comprises two or more fluid containers, each fluid container comprising at least one reservoir configured for holding a sprayable liquid, at least one interface in fluid communication with the reservoir, at least one manifold comprising at least one fluid path in fluid communication with the interface, and a common nozzle comprising at least one spray site. The spray site is in fluid communication with at least one fluid path. Optionally, at least one fluid container comprises two or more reservoirs and two or more interfaces. Each reservoir is configured for holding a disparate sprayable liquid, and each interface is in fluid communication with one reservoir and one fluid path.

In accordance with still another embodiment of the present invention, a fluid containment system for use with an electrohydrodynamic spray device is provided. The system comprises two or more fluid containers, each fluid container comprising at least one reservoir configured for holding a sprayable liquid, at least one interface in fluid communication with the reservoir, at least one manifold comprising at least one fluid path in fluid communication with the interface, and two or more nozzles. Each nozzle comprises at least one spray site in fluid communication with at least one fluid path. Optionally, the at least one said fluid container comprises two or more reservoirs, each reservoir configured for holding a disparate sprayable liquid, and two or more interfaces, each interface in fluid communication with one reservoir and one fluid path.

In accordance with yet still another embodiment, a method of delivering a sprayable liquid to a target by means of electrohydrodynamics is provided comprising: a) loading a fluid container into an electrohydrodynamic spray device, wherein the fluid container holds a sprayable liquid, entering data on the sprayable liquid into the device; c) priming the device such that the sprayable liquid is delivered to a nozzle in fluid communication with the container; d) positioning the nozzle adjacent the target; and e) delivering the sprayable liquid to the target as a charged aerosol by activating the electrohydrodynamic spray device.

These and other features and advantages of the invention will be more fully understood from the following detailed description of the invention taken together with the accompanying drawings.

The following detailed description of the embodiments of the present invention can be best understood when read in conjunction with the following drawings, where like structure is indicated with like reference numerals and in which:

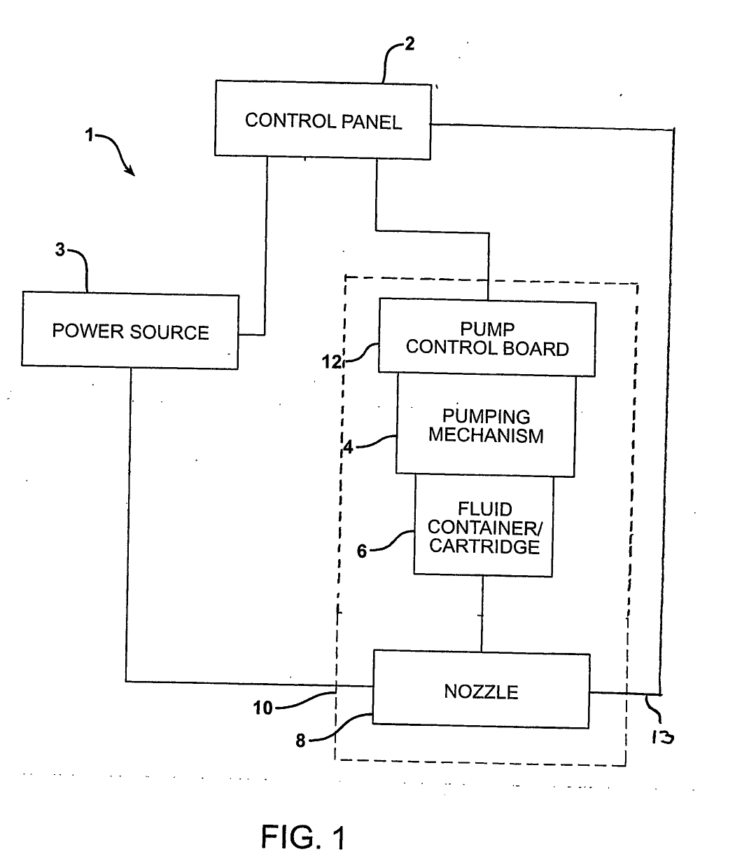

FIG. 1 is a schematic block diagram showing some of the different components that make up an electrohydrodynamic spray device comprising a fluid container in accordance with one embodiment of the present invention;

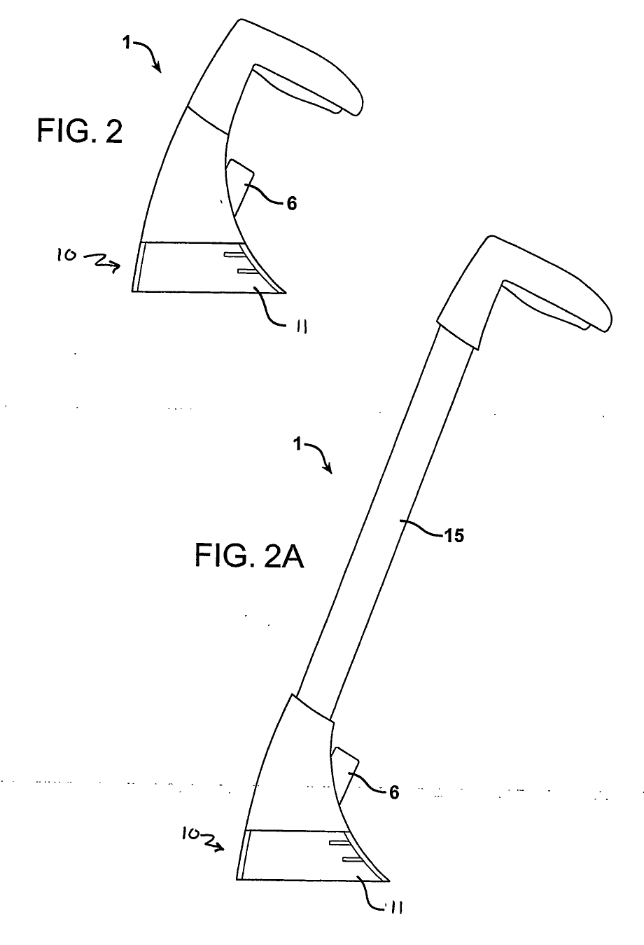

FIGS. 2 and 2A show schematic side views of an electrohydrodynamic spray device that includes a fluid container in accordance with another embodiment of the present invention;

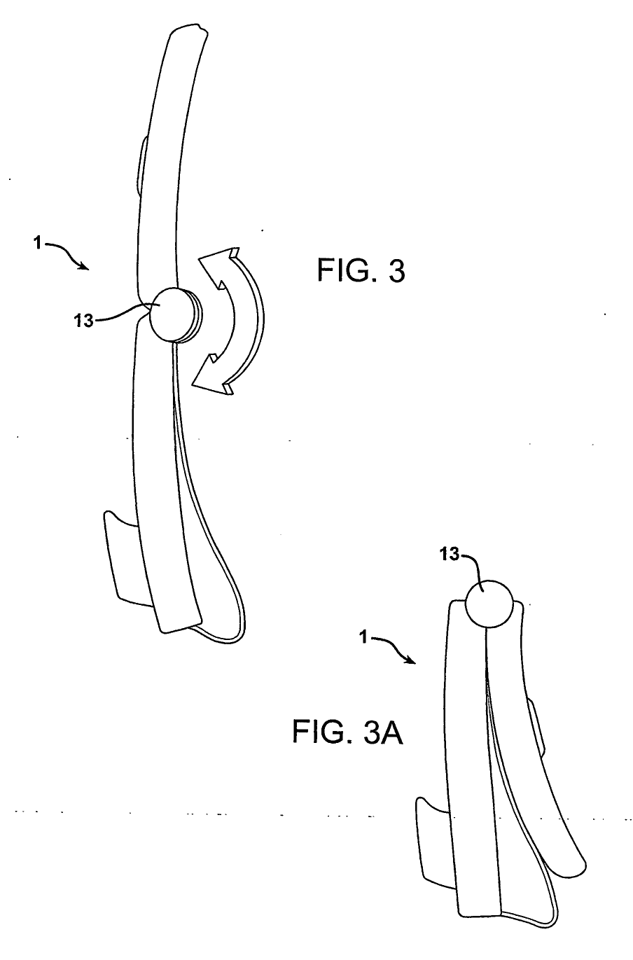

FIGS. 3 and 3A show schematic side views of an electrohydrodynamic spray device including a fluid container in accordance with still another embodiment of the present invention;

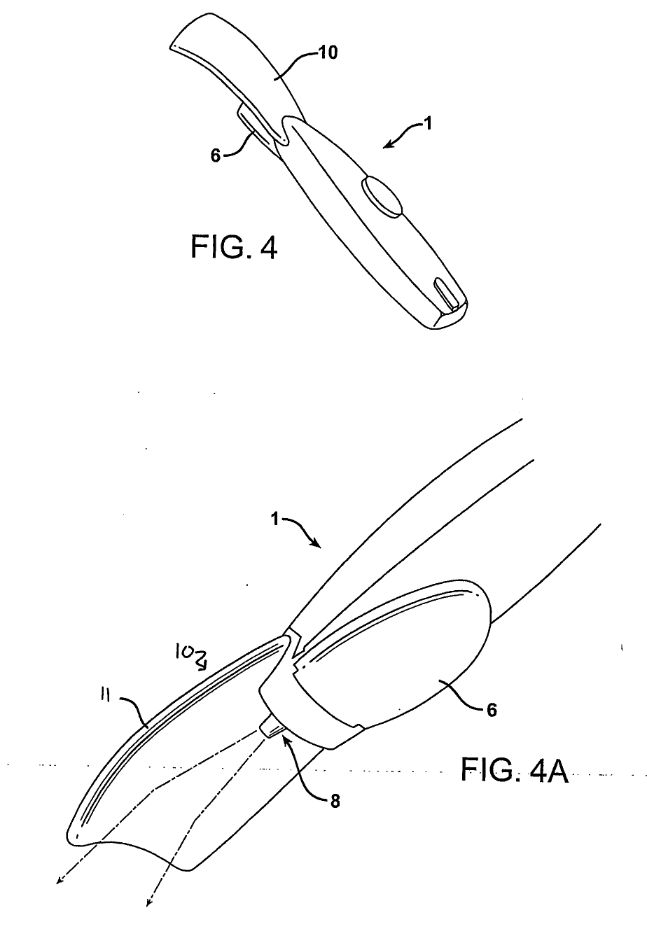

FIGS. 4 and 4A show schematic perspective views of an electrohydrodynamic spray device that includes a fluid container in accordance with yet another embodiment of the present invention;

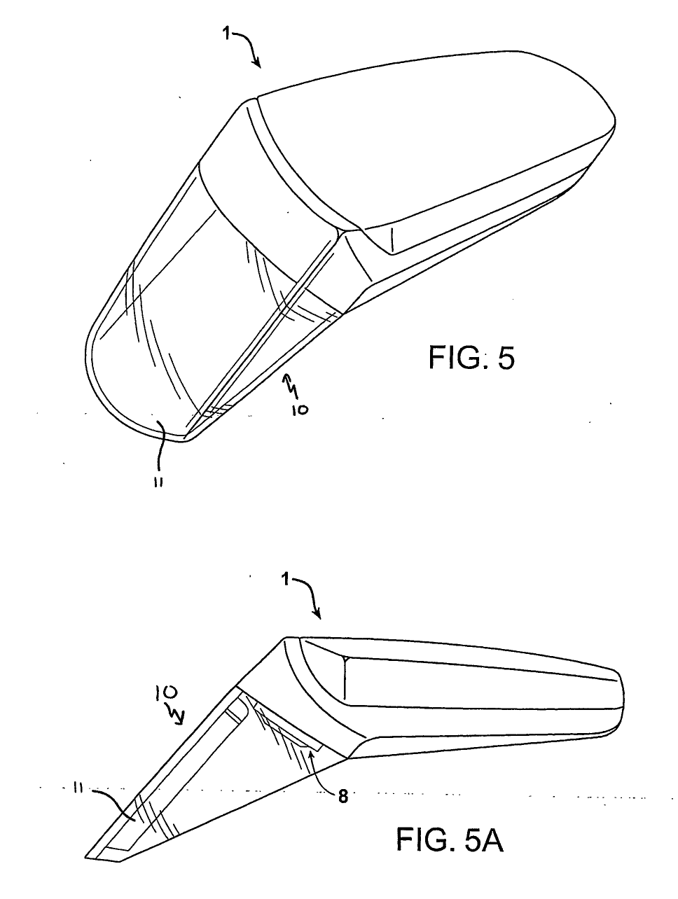

FIGS. 5 and 5A show a schematic perspective and side view, respectively, of an electrohydrodynamic spray device that includes a fluid container in accordance with yet still another embodiment of the present invention;

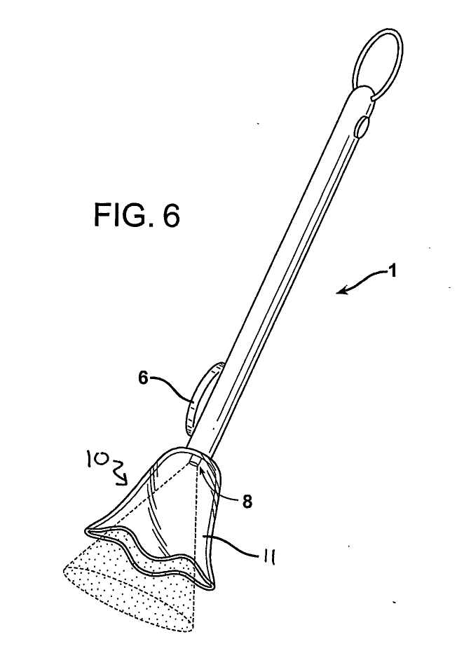

FIG. 6 shows a schematic perspective view of an electrohydrodynamic spray device that includes a fluid container in accordance with yet still another embodiment of the present invention;

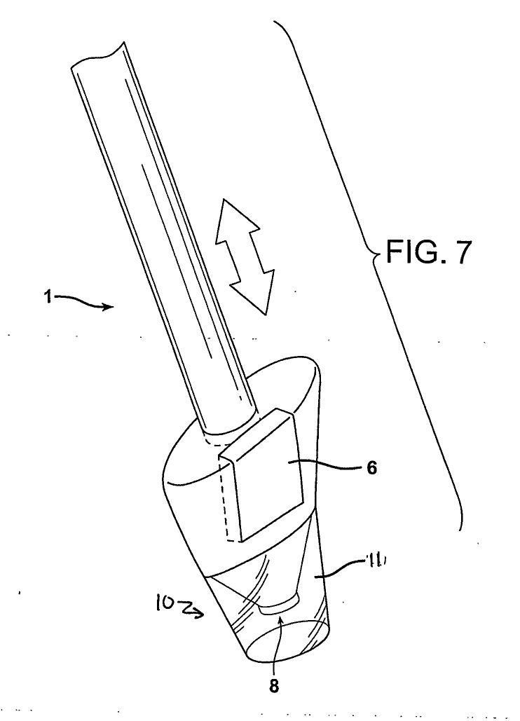

FIG. 7 shows a schematic perspective view of an electrohydrodynamic spray device that includes a fluid container in accordance with yet still another embodiment of the present invention;

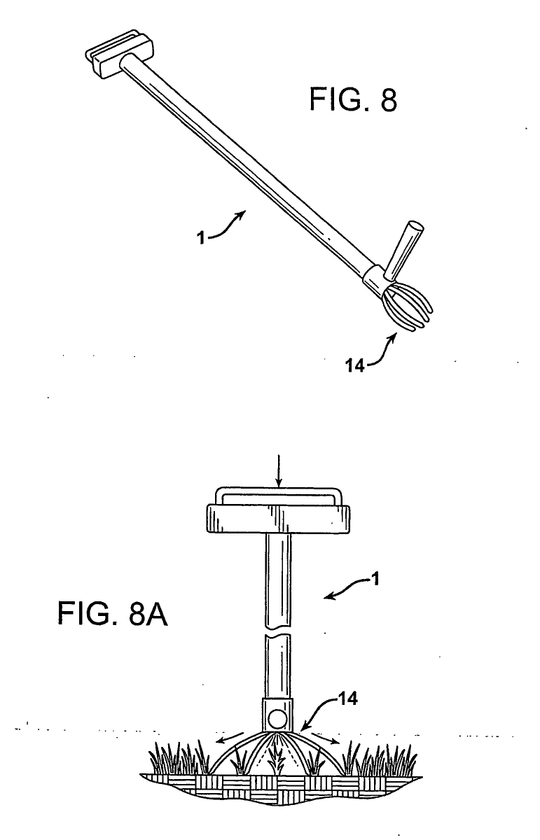

FIGS. 8 and 8A show a schematic perspective and side view, respectively, of an electrohydrodynamic spray device that includes a fluid container in accordance with yet still another embodiment of the present invention;

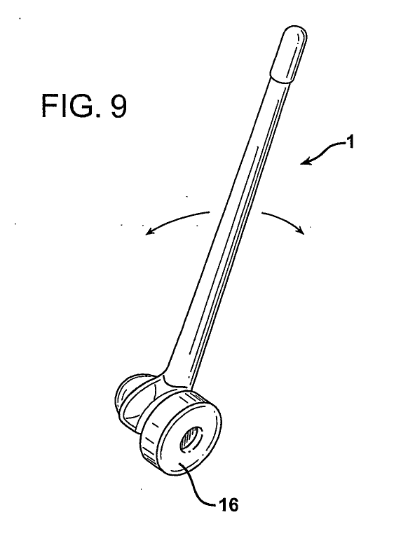

FIG. 9 shows a schematic perspective view of an electrohydrodynamic spray device that includes a fluid container in accordance with yet still another embodiment of the present invention;

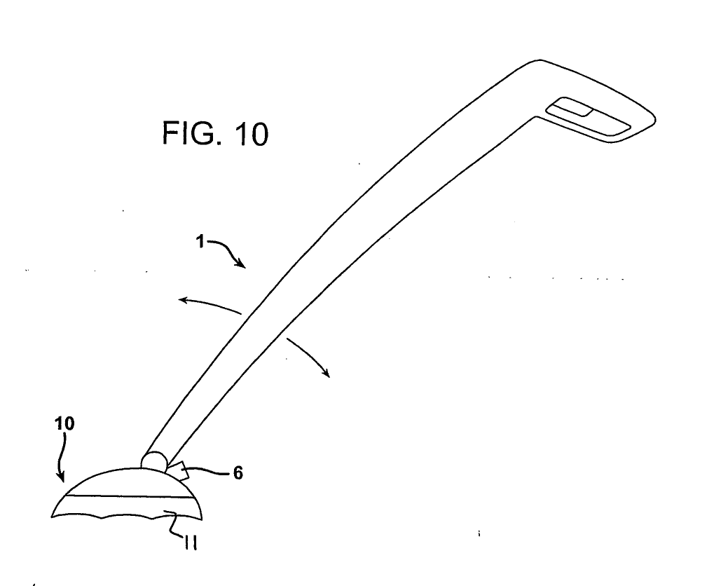

FIG. 10 shows a schematic side view of an electrohydrodynamic spray device that includes a fluid container in accordance with yet still another embodiment of the present invention; and

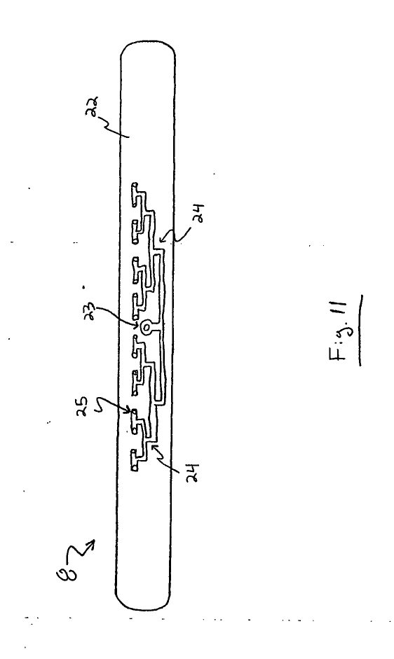

FIG. 11 shows a schematic illustration of a manifold for an electrohydrodynamic spray device in accordance with the present invention.

Skilled artisans appreciate that elements in the figures are illustrated for simplicity and clarity and have not necessarily been drawn to scale. For example, the dimensions of some of the elements in the figures may be exaggerated relative to other elements to help improve understanding of embodiment(s) of the present invention.

Referring initially to FIG. 1, a schematic block diagram of an electrohydrodynamic spray device 1 is illustrated. The device 1 can comprise the following components: a control panel 2, a power source 3, a pumping mechanism 4, a fluid container/cartridge 6, a nozzle 8, and a spray head 10. Reference is made to the following commonly assigned PCT International Patent Application entitled “SPRAY HEAD FOR ELECTROHYDRODYNAMIC SPRAY DEVICE AND ELECTROHYDRODYNAMIC SPRAYER SYSTEM” (Attorney Docket No. BAT 0079 PB/40078.256), the disclosure of which further describes the components that make up the device 1 and is incorporated herein by reference. The components can be connected by a plurality of electrical and mechanical interfaces. The device 1 can be configured to be portable and effectively provides a charged aerosol of a liquid formulation by means of electrohydrodynamics.

The control panel 2 provides an interface between the device 1 and the operator of the device 1 and can comprise one or more electrical switches for the power supply 3 and pump 4, as well as several warning LEDs for low battery and high voltage power, inter alia. The power source 3 can comprise one or more batteries, and can be configured to convert low DC voltage to the high DC voltage needed for aerosolization. By “high” DC voltage, we mean voltage in the kilovolt range. The fluid container/cartridge 6 is in fluid communication with the pumping mechanism 4 and the nozzle 8. Alternatively, the fluid container 6, pumping mechanism 4, pump control board 12, nozzle 8, or combinations thereof, can be assembled or included as a unit as part of the spray head 10. In any event, the pumping mechanism 4 is configured to pump a volume of liquid formulation from the fluid container/cartridge 6 to the nozzle 8 at a controlled flow rate, and can comprise a motion control circuit board and a DC motor (not shown), inter alia. The flow rate of the pumping mechanism 4 can be controlled by a pump control board 12. The liquid formulation can contain an active ingredient, such as a pesticidal compound.

The high voltage provided by the power source 3, pumping flow rate by way of the pump control board 12, and application time are all easily adjustable on the device 1. The device 1 in response to certain preset specifications for spraying a particular liquid formulation can make adjustments automatically. This feature contributes to the ease of use of the device 1. The user of the device 1 can also make adjustments manually. Accordingly, due to its versatility and ability to spray a wide variety of formulations, a single device 1 can be used for a wealth of different applications. For example, the device 1 can be employed for applying a pesticide to a particular target plant or animal. Since the flow rate of the formulation is controlled during a particular application, the dosage applied to the plant or animal is also very controllable, which provides for efficiency in application. Moreover, in order to configure the device 1 so that it is comfortable to use for a variety of people, as well as configured to spray a variety of sizes of plants, the size of the device 1 can also be adjustable. For example, the device 1 can be converted from a long stand-up or stick-like configuration (see FIG. 2A) to that of hand-held proportions (see FIG. 2). This conversion of the device 1 can be accomplished by many means, including folding about a rotatable joint 13 (see FIGS. 3 and 3A), removing an extension piece 15 (see FIGS. 2 and 2A), or collapsing a telescopic portion (not shown).

In accordance with one exemplary embodiment of the present invention, a fluid container 6 for use with an electrohydrodynamic spray device 1 is provided. The device 1 can be reusable, recyclable or disposable, and is configured to engage the container 6. The fluid container 6 comprises at least one reservoir configured for holding a sprayable liquid. As illustrated in FIGS. 2, 2A, 3, 3A, 4, 4A, 6, 7, 8, 8A, 9 and 10, the device 1 and/or container 6, are each configured so that when the container is engaged with the device 1, at least a portion of the container 6 extends out from the device 1 for easy access by the user for replacing with a new or different container 6, which container 6 can comprise a liquid formulation for spraying by the device 1. The arrows in FIG. 7 illustrate the changing of the fluid container 6. In accordance with the present invention, as illustrated in FIGS. 5 and 5A, the fluid container 6 can also be positioned within the handle of the device 1. However, the user can still access the container 6 in this embodiment for changing thereof. The container 6 prevents any interaction between the user of the device 1 and the fluid that it sprays, thus contributing to user safety.

The fluid container 6, further comprises at least one reservoir configured for holding a sprayable liquid, and at least one interface 23 in fluid communication with the reservoir. The fluid container 6 further comprises at least one manifold 22 comprising at least one fluid path 24. The fluid path 24 is in fluid communication with the interface. The fluid container 6 can also contain a nozzle 8 comprising at least one spray site 25 that are in fluid communication with the fluid path 24. The sprayable liquid can be selected from virtually any formulation one wishes to spray using electrohydrodynamic technology and, more particularly, can be selected from a rinsing or cleansing solution, a target preparation solution, a solution containing an active ingredient, a target sealing solution, or combinations thereof. The rinsing solution can be water or any other polar or non-polar solvent, and the target preparation solution can comprise a compound or series of compounds that, for example, open pores in the cuticle of a target (i.e., plant), such as, for example, an alkyl ester or fatty acid of other like oils (i.e., methyl oleate), which assist in the uptake and transport of certain systemic herbicidal compounds, such as, for example, a polar-acidic agrochemical complex like glyphosate, 2,4-D, glufosinate, or combinations thereof. As such, the active ingredient can be a pesticidal formulation and, more particularly, a pesticidal formulation selected from a herbicide, a fungicide, an insecticide, an acaricide, a miticide, a molluscicide, a nematicide, a rodenticide, a plant desiccant, a plant-growth regulator, or combinations thereof. The solution containing an active ingredient can be oil- or water-based, and can include the non-aqueous, oil-continuous microemulsions described in the PCT International Patent Application identified by Attorney Docket No. BAT 0080 PB/13894, the disclosure of which is incorporated herein by reference. The target sealing solution can comprise a compound or series of compounds that seal the pores of a target (i.e., plant) to seal in the active ingredient(s) and protect it from being compromised by natural or other environmental influences, such as, for example wind, (precipitation, such as rain, hail, ice) dust or irrigation. Such compound or series of compounds that seal the pores of the target can include film formers, preferably, low-cost film formers such as, for example, bone glue, polyvinyl alcohol, polyvinyl acetone, drying oils, and natural oils.

The electrohydrodynamic spray device 1 and fluid container 6 can define a security feature relationship, such that the device 1 prevents the use of an unauthorized fluid container or cartridge 6 that contains a certain sprayable liquid formulation. For example, the device 1 can be configured so that it only operates (sprays) when a container 6 containing a herbicidal formulation is engaged with the device 1. Accordingly, in this configuration, if a user inserts a container 6 containing, for example, an insecticidal formulation, the device 1 will not operate. As a result, the user cannot accidentally or unintentionally apply the wrong formulation to a target.

As illustrated in FIG. 1, the device 1 can further comprise a container/contents recognition feedback loop 13, which enables the passage of data via electrical signals from the container 6 to the control panel 2. It is contemplated that the control panel 2 functions both as a user interface and a controller that converts the user interface into appropriate electrical signals, which are in communication with the power source 3, and pump control board 12, etc. These electrical connections represent the input from the container 6 to the device 1 that the control panel 2 employs, alone or in combination with user input, to create electrical signals to the power source 3, and/or pump control board 12, which signals can vary depending upon the particular input from the container 6. As such, the fluid container 6 can also be configured so that certain formulations contained within the container 6 are recognized by the electrohydrodynamic spray device 1 causing settings such as voltage, flow rate, and spray application time, inter alia, to automatically adjust in accordance with preset specifications for certain liquid formulations. This feature enables a user to operate the device 1 without having to change settings or time the application of a particular liquid formulation, thereby contributing to the ease of use of the present invention. The automatic adjustment of spray settings and delivery time further assists to ensure that a quality spray is produced, as well as delivery of a prescribed dose of a particular liquid formulation to a particular intended target.

In addition, the fluid container or cartridge 6 can be configured so that the amount of liquid formulation contained within the container 6 can be measured by the electrohydrodynamic spray device 1, which device 1 then alerts the user when the liquid formulation is near to being depleted and a new fluid container 6 is needed. Moreover, the fluid container or cartridge 6 can be configured so that the expiration date of a certain liquid formulation contained within the container 6 can be determined by the spray device 1, which device 1 then alerts the user when the particular formulation is expired and a new fluid container or cartridge 6 is needed.

As shown in FIG. 11, the container 6 can further comprise at least one fluid path 24, at least one manifold 22 in fluid communication with the fluid path 24, and at least one nozzle 8. The nozzle 8 comprises a plurality of spray sites 25 that are in fluid communication with the manifold 22. The fluid container 6 further comprises at least one reservoir configured for holding a sprayable liquid, and at least one interface 23 in fluid communication with the reservoir.

In accordance with another embodiment of the present invention, the container 6 device 1 can comprise a single fluid path 24, a manifold 22 in fluid communication with the fluid path 24, and a common nozzle 8. The nozzle 8 comprises at least one plurality of spray site 25 in fluid communication with the fluid path 24. The container 6 comprises a reservoir configured for holding a sprayable liquid, as defined herein, and an interface 23 which is in fluid communication with the reservoir and the fluid path 14. In this configuration, individual containers 6 are employed for each specific type of application, such as, for example, individual formulation containers 6 that each separately contain herbicidal, insecticidal or fungicidal compounds, inter alia. Moreover, the fluid container 6 can optionally define a separate container containing a rinsing or cleansing solution, which solution can be sprayed or pumped through the nozzle 8 before and after each use of a formulation container 6 containing an active ingredient.

The nozzle 8 and fluid container 6 can optionally define a single unit, which would eliminate the need to clean or flush the nozzle 8 after using a formulation container 6. Moreover, a series of individual fluid containers 6 can be employed to, for example, a) pre-rinse the nozzle 8 (if not coupled with the fluid container 6), prepare the target (e.g., apply a formulation which opens the pores of a plant), c) apply the active ingredient (e.g., a pesticidal formulation), d) seal the target (e.g., apply a pore sealer to the targeted plant to seal in the pesticidal formulation and protect it from being compromised by environmental influences), and e) post-rinse the nozzle 8.

In accordance with yet another embodiment of the present invention, the container 6 can comprise two or more separate fluid paths 24, two or more separate manifolds 22, each in fluid communication with one of the separate fluid paths 22, and a common nozzle 8. The common nozzle 8 can comprise a plurality of spray sites 25, each in fluid communication with one of the separate manifolds 22. It is to be understood, however, that each separate manifold 22 is in fluid communication with a plurality of spray sites 25. The container 6 can comprise two or more separate reservoirs, each configured for holding its own disparate sprayable liquid. The container 6 can also comprise two or more interfaces 23, each one in fluid communication with one of the reservoirs as well as one of the fluid paths 24. In this configuration, each container 6 can contain one or more of the following, as defined herein, in each of the two or more separate reservoirs: a) a cleaning or rinsing solution, b) a target preparation solution, c) a solution containing an active ingredient (e.g., a pesticidal formulation), and d) a target sealing solution. Where the fluid container 6 of the instant embodiment houses multiple disparate sprayable liquids in separate reservoirs, only one container needs to be employed for multiple applications and multistage application is automatic (the user does not need to change containers between treating a target and flushing the common nozzle 8). Accordingly, the nozzle 8 can be automatically cleaned before and after each use and container formulations (e.g., pesticides) can be rinsed from the container 6 at the end of the useful lifetime of the container 6 for easy disposal thereof.

In accordance with still another embodiment of the present invention, a fluid containment system is provided comprising two or more fluid containers 6, each comprising at least one fluid path 24 and at least one manifold 22, each in fluid communication with one of the fluid paths 24, and a common nozzle 8. The common nozzle 8 comprises at least one spray site 25, each in fluid communication with one of the manifolds 22. Again, it is to be understood that each manifold 22 is in fluid communication with at least one spray site 25. While two or more containers 6 can be engaged with the device 1. In accordance with this embodiment of the present invention, each container 6 will typically comprise a single reservoir configured for holding a sprayable liquid, as well as an interface 23 in fluid communication with the reservoir and one of the fluid paths 25. However, it is also contemplated that each container 6 can comprise two or more separate reservoirs, each reservoir configured for holding disparate sprayable liquids, and each reservoir being in fluid communication with one of the fluid paths 24. Accordingly, the device 1 is configured to be engaged with multiple containers 6 and thus apply a plurality of disparate sprayable liquids through a common nozzle 8. In use, the user would indicate to the device 1 which container 6 to use for a given application by operating the control panel 2 (i.e., the cleaning solution container, the target preparation solution container, the active ingredient container, or the target sealing solution container, inter alia). Accordingly, multistage application of different formulations is provided without having to change containers 6. This feature could provide a cost advantage to the preparation of individual fluid containers 6 that comprise multiple liquid formulations, (e.g., containers comprising more than one reservoir, each configured for holding disparate sprayable liquids). Moreover, in accordance with the present embodiment, the common nozzle 8 can be automatically cleaned by employing a cleaning solution container prior to and following each application of a liquid formulation containing an active ingredient.

In accordance with yet still another embodiment of the present invention, the container 6 can comprise two or more separate fluid paths 24, two or more separate manifolds 22, each manifold 22 in fluid communication with one of the fluid paths 24, and two or more nozzles 8, each comprising at least one spray sites 25 in fluid communication with one of the manifolds 22. Here too, it is to be understood that each manifold 22 is in fluid communication with at least one spray site 25. Optionally, the container 6 can comprise two or more reservoirs, each reservoir configured for holding disparate sprayable liquid, and two or more interfaces 23, each interface 23 in fluid communication with one of the reservoirs and one of the fluid paths 24.

In addition to the features and advantages of the different embodiments of the present invention that are described herein, the instant embodiment by employing dual nozzles 8, each nozzle 8 in fluid communication with one of the manifolds 22 and, therefore, a disparate sprayable liquid, can successfully encapsulate formulations during the aerosolization thereof by the device 1 in order to increase biological efficacy. Accordingly, this particular feature could allow, for example, the combining of glyphosate with other fast acting herbicidal active ingredients for quick kill visualization of undesirable vegetation. Moreover, the two or more nozzles 8, each one in fluid communication with a disparate liquid formulation, can be sprayed simultaneously to provide a broader spectrum of active ingredients to a target. This allows for greater flexibility in the design of formulations by mixing during the aerosolization process, such as, for example, the mixing of volatile formulations.

In accordance with yet still another embodiment of the present invention, a method of delivering a sprayable liquid to a target by means of electrohydrodynamics is provided comprising: a) loading a fluid container into an electrohydrodynamic spray device, wherein the fluid container holds a sprayable liquid; b) entering data on the sprayable liquid into the device; c) priming the device such that the sprayable liquid is delivered to a nozzle in fluid communication with the container; d) positioning the nozzle adjacent to the target; and e) delivering the sprayable liquid to the target as a charged aerosol by activating the electrohydrodynamic spray device. As noted herein, the fluid container can be configured so that entering data on the sprayable liquid is automatic upon loading of the fluid container into the electrohydrodynamic device. Also, priming of the device can be automatically performed upon loading of the fluid container into the device, or upon activating the device. This step can be performed by activating a pumping mechanism in the device, which pump delivers the sprayable liquid into and through a manifold to each of a plurality of spray sites in a nozzle. Accordingly, after priming is complete, there is sprayable liquid at each spray site.

In accordance with the present invention, the container can be configured so that upon insertion of the container into the cartridge housing, the container notifies the device of its contents, and the device processes the appropriate parameters for spraying the contents of that particular container. Accordingly, the device can be backward compatible for containers, even if the device is made to adjust for parameters not originally programmed. Software upgrades for the device can allow for more contents (or defined parameters) over time. In contrast, the container can be configured so that upon insertion of the container into the device, the container notifies the device of the particular parameters needed for spraying. This information can be passed by electronic, mechanical and magnetic means. As such, the containers can be backward compatible with the device.

After positioning the nozzle adjacent to the target, activating the device will deliver a dose of sprayable liquid to the target in accordance with the flow rate, activation time and voltage which is pre-set for the particular liquid being sprayed. Multiple targets can be sprayed by repeating the method steps. When spraying is complete, the fluid container can remain in the device or it can be removed. Accordingly, upon powering off the device, the sprayable liquid can be automatically drawn back into the container. Cleaning or rinsing the device (i.e., nozzle) of the sprayable liquid can be performed by a purge step, wherein a rinsing formulation (i.e., water) is pumped through the nozzle. The rinsing formulation can be contained in a separate fluid container, or in the same container that contains an active, that container comprising multiple reservoirs in accordance with the present invention.

The following table sets out some of the spraying variables which can be pre-set in the device depending upon the specific sprayable liquid formulations contained within the fluid container, the dose desired (and thus concentration of active), and manifold size.

Various Formulations

| | | | | Dose-Total | Calculated Based on Desired Dose: | | |
|---|---|---|---|---|---|---|---|
| | Basic Parameters | | | Formulated | Formulated | | |
| | Manifold | % active | Dose-Acid | Product Delivered | Product Flow | Voltage | Activation |
| | Size | ingredients | (g/m²) | (Liters/Hectare) | Rate (ml/sec.) | (volts) | Time (sec.) |
| FIELD TRIALS | 12 inch | 1%-4% | 0.1-0.17 | 3-65 | 0.14-0.29 | −12.5 to 24.1 | 12-172 |
| GREENHOUSE | 4 inch | 1.5%-4% | 0.01-0.16 | 4-109 | 0.032-0.096 | | 3-19 |

While the invention has been described by reference to certain preferred embodiments, it should be understood that numerous changes could be made within the spirit and scope of the inventive concepts described. This includes modification to the embodiment shown in sketches from hand-held devices to other configurations which include but are not limited to boomed devices pulled behind tractors, stationary-frames used to place a plant in to treat, and devices integrally installed in green-houses/glass-houses devices. Accordingly, it is intended that the invention not be limited to the disclosed embodiments, but that it have the full scope permitted by the language of the following claims.

1. A fluid container for use with an electrohydrodynamic spray device, wherein
said container comprises
at least one reservoir configured for holding a sprayable liquid,
at least one interface in fluid communication with said reservoir,
at least one manifold comprising at least one fluid path, said fluid path in fluid communication with said interface, and
at least one nozzle comprising at least one spray site, said spray site in fluid communication with said fluid path; and
said device is configured to engage said container.

2. The fluid container of claim 1, wherein said device and said container are each configured so that when said container is engaged with said device, at least a portion of said container extends out from said device.

3. The fluid container of claim 1, wherein said sprayable liquid is selected from a rinsing solution, a target preparation solution, a solution containing an active ingredient, a target sealing solution, or combinations thereof.

4. The fluid container of claim 1, wherein
said reservoir is configured for holding a first sprayable liquid and a second sprayable liquid, said first sprayable liquid comprising a solution containing an active ingredient, and said second sprayable liquid selected from a rinsing solution, a target preparation solution, a target sealing solution, or combinations thereof.

5. The fluid container of claim 3, wherein said rinsing solution comprises a polar solvent.

6. The fluid container of claim 3, wherein said rinsing solution comprises a non-polar solvent.

7. The fluid container of claim 3, wherein said rinsing solution comprises water.

8. The fluid container of claim 3, wherein said target preparation solution comprises a compound that opens pores in the cuticle of a target.

9. The fluid container of claim 8, wherein said compound that opens the pores in the cuticle of a target is an alkyl ester of fatty acid.

10. The fluid container of claim 9, wherein said alkyl ester of fatty acid is methyl oleate.

11. The fluid container of claim 3, wherein said solution containing an active ingredient is water-based.

12. The fluid container of claim 3, wherein said solution containing an active ingredient is oil-based.

13. The fluid container of claim 12, wherein said oil-based solution is a non-aqueous, oil-continuous microemulsion.

14. The fluid container of claim 3, wherein said solution containing an active ingredient is a pesticidal formulation.

15. The fluid container of claim 14, wherein said pesticidal formulation is selected from a herbicide, a fungicide, an insecticide, an acaricide, a miticide, a molluscicide, a nematicide, a rodenticide, a plant desiccant, a plant-growth regulator, or combinations thereof.

16. The fluid container of claim 15, wherein said herbicide is a polar-acidic agrochemical complex selected from glyphosate, 2,4-D, glufosinate, or combinations thereof.

17. The fluid container of claim 3, wherein said target sealing solution comprises a compound that seals the pores in the cuticle of a target.

18. The fluid container of claim 17, wherein said compound that seals the pores in the cuticle of a target is selected from bone glue, polyvinyl alcohol, polyvinyl acetate, drying oils, natural oils, and combinations thereof.

19. The fluid container of claim 1, wherein said container is configured to be refillable, recyclable, or disposable.

20. The fluid container of claim 1, wherein said container is configured to prevent any interaction between a user of said electrohydrodynamic spray device and said sprayable liquid contained within said container.

21. The fluid container of claim 1, wherein said container is configured such that when said container is engaged with said electrohydrodynamic spray device, said device recognizes said sprayable liquid contained within said container and automatically adjusts certain pre-defined settings in accordance with the characteristics of said sprayable liquid.

22. The fluid container of claim 21, wherein said pre-defined settings are entered manually by a user of said device in accordance with the characteristics of said sprayable liquid.

23. The fluid container of claim 1, wherein said container is configured such that when said container is engaged with said electrohydrodynamic spray device, said device recognizes said sprayable liquid contained within said container and allows the spraying thereof.

24. The fluid container of claim 1, wherein said container is configured such that when said container is engaged with said electrohydrodynamic spray device, said device recognizes the amount and/or expiration date of said sprayable liquid contained within said container and alerts a user of said device when a new container is required.

25. A fluid container for use with an electrohydrodynamic spray device, wherein
said container comprises
two or more separate reservoirs, each said reservoir configured for holding a disparate sprayable liquid,
two or more interfaces, each said interface in fluid communication with one said reservoir,
at least one manifold comprising two or more separate fluid paths, each said fluid path in fluid communication with one said interface, and
at least one nozzle comprising at least one spray site, said spray site in fluid communication with one or more said fluid paths; and
said device is configured to engage said container.

26. A fluid containment system for use with an electrohydrodynamic spray device, wherein said system comprises
two or more fluid containers, each said fluid container comprising
at least one reservoir configured for holding a sprayable liquid,
at least one interface in fluid communication with said reservoir,
at least one manifold comprising at least one fluid path, said fluid path in fluid communication with said interface, and
a common nozzle, said nozzle comprising at least one spray site, said spray site in fluid communication with at least one fluid path.

27. The fluid containment system of claim 26, wherein
at least one said fluid container comprises
two or more reservoirs, each said reservoir configured for holding a disparate sprayable liquid, and
two or more interfaces, each said interface in fluid communication with one said reservoir and one said fluid path.

28. A fluid containment system for use with an electrohydrodynamic spray device, wherein said system comprises
two or more fluid containers, each said fluid container comprising
at least one reservoir configured for holding a sprayable liquid,
at least one interface in fluid communication with said reservoir,
at least one manifold comprising at least one fluid path, said fluid path in fluid communication with said interface, and
two or more nozzles, each said nozzle comprising at least one spray site, said spray site in fluid communication with at least one fluid path.

29. The fluid containment system of claim 26, wherein
at least one said fluid container comprises
two or more reservoirs, each said reservoir configured for holding a disparate sprayable liquid, and
two or more interfaces, each said interface in fluid communication with one said reservoir and one said fluid path.

30. A method of delivering a sprayable liquid to a target by means of electrohydrodynamics comprising:
a) loading a fluid container into an electrohydrodynamic spray device, wherein said fluid container holds a sprayable liquid;
b) entering data on said sprayable liquid into said device;
c) priming said device such that said sprayable liquid is delivered to a nozzle in fluid communication with said container;
d) positioning said nozzle adjacent said target; and
e) delivering said sprayable liquid to said target as a charged aerosol by activating said electrohydrodynamic spray device.

31. The method of claim 30, wherein said electrohydrodynamic spray device further comprises a microprocessor and said entering data on said sprayable liquid into said device is automatically performed upon loading said fluid container into said electrohydrodynamic device.

32. The method of claim 30, wherein said priming is automatically performed upon loading said fluid container into said electrohydrodynamic spray device.

33. The method of claim 30 further comprising rinsing said nozzle prior to and/or following said delivering said sprayable liquid.

34. A method of delivering a sprayable liquid to a target by means of electrohydrodynamics comprising:
a) loading a fluid container into an electrohydrodynamic spray device, wherein said fluid container holds a sprayable liquid;
b) entering data on said sprayable liquid into said device; and
c) adjusting certain pre-defined spray parameters in accordance with the characteristics of said sprayable liquid and said data entered.

35. The method of claim 34 wherein said entering data on said sprayable liquid and said adjusting certain pre-defined spray parameters is automatically performed by said device or manually entered by a user.
